(19) United States
(12) Patent Application Publication
(10) Pub. No.: US 2002/0189034 A1
(43) Pub. Date: Dec. 19, 2002

(54) Primary agent for two-agent hair dyeing/bleaching composition, two-agent hair dyeing/bleaching composition kit, and hair treatment method using it

(75) Inventors: Yasuhiko Kitabata, Fukui-shi (JP); Haruhiko Yoshida, Fukui-shi (JP); Teiji Izawa, Fukui-shi (JP); Hitomi Kameoka, Fukui-shi (JP)

Correspondence Address: LEYDIG VOIT & MAYER, LTD, 700 THIRTEENTH ST. NW, SUITE 300, WASHINGTON, DC, 20005-3960, US

(73) Assignee: NICCA CHEMICAL CO., LTD., Fukui (JP)

(21) Appl. No.: 10/157,188

(22) Filed: May 30, 2002

(30) Foreign Application Priority Data
May 31, 2001 (JP) 2001-165420

(51) Int. Cl. A61K 7/13

(52) U.S. Cl. 8/405; 8/406; 8/408

(57) ABSTRACT

A primary agent for a two-agent hair dyeing/bleaching composition which comprises a primary agent containing an alkali agent and a secondary agent containing an oxidizing agent, wherein the primary agent contains:

(a) at least one alkali agent selected from the group consisting of ammonia water and monoethanolamine,

(b) at least one water-soluble ammonium salt selected from the group consisting of ammonium carbonate and ammonium hydrogencarbonate, and

(c) at least one first pH adjustor selected from the group consisting of polycarboxylic acids and their salts.

### BACKGROUND OF THE INVENTION

1. Field of the Invention

The present invention relates to a primary agent for a two-agent hair dyeing/bleaching composition for dyeing or bleaching of hair, to a two-agent hair dyeing/bleaching composition kit, and to a hair treatment method using it.

2. Related Background Art

A strong demand exists in the market for dyeing hair to a lighter color tint than the original hair color, and numerous bleaching and dyeing agents are on the market. Particularly in recent years there has been a preference for even lighter color tints, and therefore for methods of dyeing while bleaching even more of the melanin pigment originally present in the hair.

As “bleaching agents” used for this purpose there are known, for example, two-agent oxidative bleaching agent compositions comprising a primary agent containing an alkali agent and a secondary agent containing an oxidizing agent such as hydrogen peroxide. The primary and secondary agents are mixed just before use for oxidative bleaching of the melanin pigment, and the reaction is promoted by the oxidizing agent (hydrogen peroxide, etc.) which is activated by the alkali agent.

As “dyeing agents” there are known, for example, two-agent oxidative dyeing agent compositions comprising a primary agent containing an alkali agent and an oxidizing dye intermediate such as paraphenylenediamine and a secondary agent containing an oxidizing agent such as hydrogen peroxide. The primary and secondary agents are mixed just before use for dyeing while simultaneously accomplishing oxidative bleaching of the melanin pigment and production of a dye (hair dyeing) by oxidative polymerization of the oxidizing dye intermediate, and these reactions are also carried out by the oxidizing agent (hydrogen peroxide, etc.) which is activated by the alkali agent.

Ammonia water and monoethanolamine have conventionally been used as alkali agents to impart sufficient bleaching and dyeing power to the hair dyeing/bleaching composition. However, these have had a major drawback since the use of ammonia water and monoethanolamine is associated with sharp odor or skin irritation, thus producing discomfort when applied by consumers.

As alternatives to the use of ammonia water there have been disclosed methods of mixing in low-odor organic amines (Japanese Patent Application Laid-Open Gazette No. HEI 1-213220, Japanese Patent Application Laid-Open Gazette No. HEI 5-246827 and Japanese Patent Application Laid-Open Gazette No. 2000-351718), or methods of including guanidium salts (Japanese Patent Application Laid-Open Gazette No. HEI 10-25230 and Japanese Patent Application Laid-Open Gazette No. HEI 11-180837).

However, organic amines and guanidium salts do not exhibit adequate bleaching power and thus do not produce dyeing to a very light color tint. They also tend to remain on the hair, causing significant hair damage or problems of dryness and fading, while also failing to reduce scalp irritation.

Japanese Patent Application Laid-Open Gazette No. SHO 60-155108 discloses a dyeing/bleaching composition employing both ammonia and a water-soluble ammonium salt which exhibits excellent bleaching power at relatively low pH and allows deep dyeing, but this dyeing/bleaching composition is also inadequate in terms of alleviating scalp irritation.

### SUMMARY OF THE INVENTION

It is an object of the present invention, which has been accomplished in light of the aforementioned problems of the prior art, to provide a primary agent for a two-agent hair dyeing/bleaching composition comprising a primary agent containing an alkali agent and a secondary agent containing an oxidizing agent, wherein the primary agent exhibits excellent hair bleaching and dyeing effects with adequately reduced scalp irritation, as well as a two-agent hair dyeing/bleaching composition kit and a hair treatment method in which it is employed.

As a result of much diligent research directed toward achieving the aforementioned object, the present inventors have completed the present invention upon finding that by using a specific composition as the primary agent for a two-agent hair dyeing/bleaching composition, excellent bleaching and dyeing effects are exhibited while scalp irritation is sufficiently alleviated.

The primary agent for a two-agent hair dyeing/bleaching composition according to the invention comprises a primary agent containing an alkali agent and a secondary agent containing an oxidizing agent, the primary agent for a two-agent hair dyeing/bleaching composition containing:

(a) at least one alkali agent selected from the group consisting of ammonia water and monoethanolamine,

(b) at least one water-soluble ammonium salt selected from the group consisting of ammonium carbonate and ammonium hydrogencarbonate, and

(c) at least one first pH adjustor selected from the group consisting of polycarboxylic acids and their salts.

The two-agent hair dyeing/bleaching composition kit according to the invention is provided with

(i) the primary agent for a two-agent hair dyeing/bleaching composition of the invention, and

(ii) a secondary agent containing an oxidizing agent.

The hair treatment method according to the invention is a method of applying to hair a hair dyeing/bleaching composition comprising

(a) at least one alkali agent selected from the group consisting of ammonia water and monoethanolamine,

(b) at least one water-soluble ammonium salt selected from the group consisting of ammonium carbonate and ammonium hydrogencarbonate,

(c) at least one first pH adjustor selected from the group consisting of polycarboxylic acids and their salts, and

(e) an oxidizing agent.

In the primary agent for a two-agent hair dyeing/bleaching composition, the two-agent hair dyeing/bleaching composition kit and the hair treatment method using it according to the invention, the polycarboxylic acids and their salts of component (c) are preferably citric acid, tartaric acid, malic acid and their salts.

The primary agent for a two-agent hair dyeing/bleaching composition according to the invention preferably contains the alkali agent of component (a) at 1-25 wt % (where the ammonia water weight is expressed in terms of ammonia water at a concentration of 28 wt %), the water-soluble ammonium salt of component (b) at 0.5-20 wt % and the first pH adjustor of component (c) at 0.1-10 wt %.

The primary agent for a two-agent hair dyeing/bleaching composition and the hair dyeing/bleaching composition of the invention preferably further contains (d) at least one second pH adjustor selected from the group consisting of sodium hydroxide, potassium hydroxide and sodium metasilicate, and the content of the second pH adjustor of component (d) is preferably 0.1-8 wt %.

The pH of the primary agent for a two-agent hair dyeing/bleaching composition of the invention is preferably 9.0-12.0, and the pH of the hair dyeing/bleaching composition of the invention is also preferably 9.0-12.0.

Incidentally, hair dyeing/bleaching agents in a wide sense include “dyeing agents” which dye hair and “bleaching agents” which bleach hair, and the term “hair dyeing/bleaching composition” according to the invention will be used to indicate both.

The present invention will become more fully understood from the detailed description given hereinbelow and the accompanying drawings which are given by way of illustration only, and thus are not to be considered as limiting the present invention.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DESCRIPTION OF THE PREFERRED EMBODIMENTS

The primary agent for a two-agent hair dyeing/bleaching composition according to the invention will now be explained. The primary agent of the invention comprises

(a) at least one alkali agent selected from the group consisting of ammonia water and monoethanolamine,

(b) at least one water-soluble ammonium salt selected from the group consisting of ammonium carbonate and ammonium hydrogencarbonate, and

(c) at least one first pH adjustor selected from the group consisting of polycarboxylic acids and their salts, and it is used as the primary agent for a two-agent hair dyeing/bleaching composition comprising a primary agent containing an alkali agent and a secondary agent containing an oxidizing agent.

According to the invention, the content of the alkali agent of component (a) in the primary agent is preferably 1-25 wt % and more preferably 3-20 wt %. The ammonia water weight is expressed in terms of ammonia water with an ammonia concentration of 28 wt %. When the content of component (a) is less than 1 wt %, the bleaching effect is reduced and lighter dyeing becomes difficult to achieve. On the other hand, when the content of component (a) is greater than 25 wt %, not only is a correspondingly greater bleaching effect not achieved, but scalp irritation tends to be stronger and hair damage more extensive.

According to the invention, the content of the water-soluble ammonium salt of component (b) in the primary agent is preferably 0.5-20 wt % and more preferably 1-15 wt %. When the content of component (b) is less than 0.5 wt %, the effect of preventing scalp irritation tends to be poor, and when the content of component (b) is greater than 20 wt %, the bleaching and dyeing effects tend to be reduced.

According to the invention, a polycarboxylic acid or its salt is used as a first pH adjustor for component (c). Using a polycarboxylic acid or its salt as the pH adjustor can adequately alleviate scalp irritation, whereas scalp irritation is not adequately alleviated with monocarboxylic acids or their salts.

There are no particular restrictions on the polycarboxylic acids so long as they are carboxyl acids commonly used as pH adjustors in cosmetics, and as examples there may be mentioned adipic acid, azelaic acid, citric acid, fumaric acid, ketoglutaric acid, maleic acid, malic acid, malonic acid, oxalic acid, sebacic acid, succinic acid, tartaric acid, aspartic acid and glutamic acid. Salts of these polycarboxylic acids may also be used without restrictions so long as they are physiologically acceptable, and as examples there may be mentioned salts of alkali metals such as sodium and potassium, salts of alkaline earth metals such as calcium and magnesium, ammonium salts or salts of organic amines such as monoethanolamine or triethanolamine, and salts of basic amino acids such as arginine or lysine. The use of one or more selected from the group consisting of malic acid, tartaric acid, citric acid and salts thereof for component (c) is preferred for a particularly excellent hair bleaching and dyeing effect, and for adequately reduced scalp irritation.

The content of the first pH adjustor as component (c) in the primary agent is preferably 0.1-10 wt % and more preferably 0.2-9 wt %. When the content of component (c) is less than 0.1 wt %, the effect of preventing scalp irritation tends to be poor, and when the content of component (c) is greater than 10 wt %, the bleaching and dyeing effects tend to be reduced.

The primary agent for a two-agent hair dyeing/bleaching composition according to the invention also preferably comprises (d) at least one second pH adjustor selected from the group consisting of sodium hydroxide, potassium hydroxide and sodium metasilicate, in addition to components (a), (b) and (c) described above. Adding such a second pH adjustor will allow fine adjustment of the pH of the primary agent of the invention. When component (d) is included, its content is preferably 0.1-8 wt % and more preferably 0.1-6 wt %. If the content of component (d) is less than 0.1 wt % it will tend to be difficult to achieve fine adjustment of the pH of the primary agent, whereas if the content of component (d) is greater than 8 wt %, scalp irritation may tend to be stronger and hair damage more extensive.

According to the invention, the pH of the primary agent for a two-agent hair dyeing/bleaching composition is preferably 9.0-12.0 and more preferably 9.1-11.8. A pH of greater acidity than 9.0 will tend to give a poor bleaching effect and also reduce the dyeing effect. On the other hand, a pH of greater alkalinity than 12.0 will tend to result in stronger scalp irritation and more extensive hair damage.

When the primary agent for a two-agent hair dyeing/bleaching composition according to the invention is to be used for a “dyeing agent ”, it may contain a hair dye in addition to the above-mentioned components. When it is to be used for a “bleaching agent”, it is not necessary to include a hair dye.

As hair dyes there may be mentioned dyes commonly used for hair dyeing, such as oxidizing dye intermediates, oxidizing dye couplers, direct dyes and the like. As examples of such dyes there may be mentioned those listed in the Guidelines for Hair Dye Production Approval Application (Revised 5th Printing) published by the Japan Hair Color Association, and specifically, 4-amino-2-hydroxytoluene, 2-amino-4-nitrophenol, 2-amino-5-nitrophenol, 1-amino-4-methylaminoanthraquinone, 3,3′-iminodiphenol, 2,4-diaminophenoxyethanol HCl, 2,4-diaminophenol HCl, Toluene-2,5-diamine hydrochloride, 2-nitro-p-phenylenediamine dihydrochloride, p-phenylenediamine HCl, N-phenyl-p-phenylenediamine HCl, m-phenylenediamine hydrochloride, o-aminophenol, Pyrocatechol, N-phenyl-p-phenylenediamine acetate, 1,4-diaminoanthraquinone, 2,6-diaminopyridine, 1,5-naphthalenediol, Diphenylamine, Tannic acid, Toluene-2,5-diamine, Toluene-3,4-diamine, 1-naphthol, 2-nitro-p-phenylenediamine, p-aminophenol, 4-nitro-o-phenylenediamine, p-phenylenediamine, p-methylaminophenol, Picramic acid, Sodium picramate, N,N′-bis(4-aminophenol)-2,5-diamino-1,4-quinonediimine, 2-methyl-5-hydroxyethylaminophenol, Hydroquinone, Pyrogallol, N-phenyl-p-phenylenediamine, Phloroglucin, Haematoxylon campechianum wood extract, Gallic acid, m-aminophenol, m-phenylenediamine, 5-amino-o-cresol sulfate, 2-amino-5-nitrophenol sulfate, o-aminophenol sulfate, 2-chloro-p-phenylenediamine sulfate, 4,4′-diaminodiphenylamine sulfate, 2,4-diaminophenol sulfate, Toluene-2,5-diamine sulfate, 2-nitro-p-pheylenediamine sulfate, p-aminophenol sulfate, 4-nitro-o-phenylenediamine sulfate, p-nitro-m-phenylenediamine sulfate, p-phenylenediamine sulfate, p-methylaminophenol sulfate, m-aminophenol sulfate, p-phenylenediamine sulfate and Resorcinol. These may be used alone or in combinations of two or more.

In consideration of the hair dyeing effect, the content of the hair dye in the primary agent is preferably 0.0001-20 wt % and more preferably 0.001-10 wt %.

Whether the purpose is for a “dyeing agent” or a “bleaching agent”, the primary agent for a two-agent hair dyeing/bleaching composition according to the invention may also contain dyes known as tar pigments for cosmetics, in order to further ajust the color tone.

As tar pigments for cosmetics there may be mentioned the legal pigments listed in the Handbook of Legal Pigments, edited by the Japan Cosmetic Industry Association, such as (Brown) C.I. 20170; (Black) C.I. 20470; (Violet) D & C Violet No.2, C.I. 60730; (Blue) FD & C Blue No.1, FD & C Blue No.2, C.I. 73000, C.I. 42052, D & C Blue No.9, D & C Blue No.4, C.I. 61520, C.I. 74160; (Green) D & C Green No.5, D & C Green No.6, D & C Green No.8, C.I. 42095, FD & C Green No.3, C.I. 10020, C.I. 42085; (Yellow) D & C Yellow No.7, D & C Yellow No.8, D & C Yellow No.10, D & C Yellow No.11, C.I. 21090, FD & C Yellow No.5, C.I. 11680, C.I. 18950, C.I. 10316, C.I. 11380, C.I. 11390, C.I. 13065, C.I. 18820, FD & C Yellow No.6; (Orange) D & C Orange No.5, C.I. 12075, C.I. 21110, D & C Orange No.4, D & C Orange No.10, D & C Orange No.11, C.I. 11725, C.I. 14600, C.I. 12100; (Red) C.I. 16255, D & C Red No.28, C.I. 45440, C.I. 45100, C.I. 16185, D & C Red No.6, D & C Red No.7, C.I. 15585, C.I. 15630, C.I. 45170, D & C Red No.27, D & C Red No.31, D & C Red No.34, C.I. 12120, D & C Red No.21, D & C Red No.17, D & C Red No.30, D & C Red No.33, C.I. 12085, D & C Red No.22, C.I. 45380, C.I. 45410, C.I. 45440, FD & C Red No.3, C.I. 45190, C.I. 12315, C.I. 15865, C.I. 26105, C.I. 16155, C.I. 16150, FD & C Red No.4, C.I. 12140 and C.I. 15620.

When these tar pigments for cosmetics are used, their content in the primary agent is preferably 0.00001-20 wt % and more preferably 0.0001-10 wt %.

The primary agent for a two-agent hair dyeing/bleaching composition according to the invention may also contain other appropriately selected components that may normally be included in bleaching agents or dyeing agents, for example, oily components, solubilizers, emulsifiers, hair protecting components such as hydrolysates of collagen, gelatin, etc., and derivatives thereof, as well as humectants, stabilizers, thickeners, solvents such as ethanol, chelating agents, preservatives, ultraviolet absorbers, aromatics and the like, within a range that does not hamper the effect of the invention.

As oily components there may be mentioned, for example, oils and fats such as paraffin, liquid paraffin, bees wax, squalane, jojoba oil, olive oil, ester oils, triglycerides, vaseline and lanolin; silicone derivatives such as dimethylpolysiloxane, methylphenylpolysiloxane, amino-modified silicone oil, alcohol-modified silicone oil, fluorine-modified silicone oil, polyether-modified silicone oil and alkyl-modified silicone oil; higher fatty acids such as lauric acid, myristic acid, palmitic acid, stearic acid, behenic acid, oleic acid, undecylic acid and isostearic acid; and higher alcohols such as lauryl alcohol, cetanol, stearyl alcohol, cetostearyl alcohol, oleyl alcohol and behenyl alcohol. These oily components may be used alone or in combinations of two or more, and their content in the primary agent is preferably 0.1-50 wt %.

As the aforementioned solubilizers and emulsifiers there may be appropriately used ether-based nonionic surfactants, anionic surfactants, cationic surfactants and amphoteric surfactants, such as polyoxyethylene oleyl ether, polyoxyethylene stearyl ether, polyoxyethylene cetyl ether, polyoxyethylene lauryl ether, polyoxyethylene octylphenyl ether, polyoxyethylene dodecylphenyl ether and polyoxyethylene nonylphenyl ether. These solubilizers and emulsifiers may be used alone or in combinations of two or more, and their content in the primary agent is preferably 0.1-40 wt %.

As the aforementioned humectants there may be mentioned, for example, glycerin, diglycerin, propylene glycol, dipropylene glycol, 1,3-butyleneglycol, sorbitol, plant extracts, vitamins, hyaluronic acid salts, chondroitin sulfate, and the like. As the aforementioned stabilizers there may be mentioned sulfurous acid salts, thioglycolic acid salts, cysteines, ascorbic acid, and the like. As the aforementioned thickeners there may be mentioned carboxymethyl cellulose, carboxyvinyl polymer, hydroxyethyl cellulose, hydroxypropyl cellulose, xanthan gum, carrageenan, alginic acid salts, pectins, tragacanth gum, and the like.

There are no particular restrictions on the method of producing the primary agent for a two-agent hair dyeing/bleaching composition according to the invention, and for example, it may be obtained by combining prescribed amounts of the components to be combined together with a solvent or dispersing medium such as purified water, by a conventional publicly known method. The form thereof is also not particularly restricted, and any of various forms such as a liquid, foam, gel, cream, paste or the like may be used.

A two-agent hair dyeing/bleaching composition kit according to the invention will now be explained. The two-agent hair dyeing/bleaching composition kit of the invention is provided with

(i) the primary agent for a two-agent hair dyeing/bleaching composition of the invention, and

(ii) a secondary agent containing an oxidizing agent.

As oxidizing agents to be contained in the secondary agent there may be mentioned hydrogen peroxide, urea peroxide, sodium percarbonate, perboric acid salts, peroxidated pyrophosphates and the like, among which hydrogen peroxide is preferred. The content of the oxidizing agent in the secondary agent is preferably 1-12 wt % and more preferably 1-6 wt %. When the oxidizing agent content is less than 1 wt %, the bleaching effect is reduced and light dyeing becomes difficult to achieve. On the other hand, when the content of the oxidizing agent is greater than 12 wt %, not only is a correspondingly greater bleaching effect not achieved, but scalp irritation tends to be stronger and hair damage more extensive.

The secondary agent according to the invention is not particularly restricted so long as it contains the aforementioned oxidizing agent, and it may also contain other appropriately selected components, for example, stabilizers such as phenacetin, oxyquinoline sulfate, salicylic acid, sodium stannate and acetanilide; pH adjustors such as phosphoric acid, pyrophosphoric acid and sodium pyrophosphate; higher alcohols such as cetanol, stearyl alcohol, cetostearyl alcohol, oleyl alcohol and behenyl alcohol; surfactants such as sodium lauryl sulfate, sodium polyoxyethylene lauryl ether sulfate, cetyl trimethylammonium chloride, stearyl trimethylammonium chloride, cetyl trimethylammonium bromide, stearyl trimethylammonium bromide, polyoxyethylene cetyl ether and polyoxyethylene oleyl ether; chelating agents such as disodium edetate, pentasodium diethylenetriamine pentaacetate and hydroxyethanediphosphonic acid; as well as aromatics, humectants, hair protecting components, preservatives, and the like, within a range that does not hamper the effect of the invention.

The pH of the secondary agent of the invention is preferably 1.5-6 and more preferably 2-5.5. A pH outside of this range will tend to result in poor shelf life of the oxidizing agent and hence render it difficult to preserve quality.

There are no particular restrictions on the method of producing the secondary agent of the invention, and for example, it may be obtained by combining prescribed amounts of the components to be combined together with a solvent or dispersing medium such as purified water, by a conventional publicly known method. The form thereof is also not particularly restricted, and any of various forms such as a liquid, foam, gel, cream, paste or the like may be used.

The mixing ratio of the primary agent and secondary agent according to the invention is not particularly restricted but is preferably in the range of 1:0.5-1:3 and more preferably in the range of 1:0.8-1:2, in terms of weight ratio.

The hair treatment method of the invention will now be explained. The hair treatment method of the invention is a method of applying to hair a hair dyeing/bleaching composition comprising

(a) at least one alkali agent selected from the group consisting of ammonia water and monoethanolamine,

(b) at least one water-soluble ammonium salt selected from the group consisting of ammonium carbonate and ammonium hydrogencarbonate,

(c) at least one first pH adjustor selected from the group consisting of polycarboxylic acids and their salts, and

(e) an oxidizing agent.

The hair dyeing/bleaching composition of the invention comprises the aforementioned components (a), (b), (c) and (e), which are described above. The preferred contents for each of the components in the hair dyeing/bleaching composition of the invention are component (a): 0.5-12.5 wt % (where the ammonia water weight is expressed in terms of ammonia water at a concentration of 28 wt %), component (b): 0.25-10 wt %, component (c): 0.05-5 wt % and component (e): 0.5-6 wt %.

The hair dyeing/bleaching composition according to the invention also preferably comprises (d) at least one second pH adjustor selected from the group consisting of sodium hydroxide, potassium hydroxide and sodium metasilicate, and the content of component (d) in the hair dyeing/bleaching composition is preferably 0.05-4 wt %.

The pH of the hair dyeing/bleaching composition according to the invention is preferably 9.0-12.0 and more preferably 9.1-11.8. A pH of greater acidity than 9.0 will tend to give a poor bleaching effect and also reduce the dyeing effect. On the other hand, a pH of greater alkalinity than 12.0 will tend to result in stronger scalp irritation and more extensive hair damage.

The hair dyeing/bleaching composition according to the invention may be satisfactorily obtained by mixing the primary and secondary agents according to the invention, and the resulting composition may be used as a dyeing agent or bleaching agent. Incidentally, the components mentioned above for inclusion in the primary agent may also be added to the secondary agent so long as they are components that are stable with respect to the oxidizing agent in the secondary agent, such as, for example, (c) polycarboxylic acids and their salts.

There are no particular restrictions on the specific method of applying the hair dyeing/bleaching composition to hair for the hair treatment method of the invention, and for example, the hair dyeing/bleaching composition of the invention may be coated onto hair, allowed to stand for a prescribed period of time and then rinsed off, for treatment of hair, i.e. hair bleaching or dyeing, with an adequately reduced degree of scalp irritation.

### EXAMPLES

The present invention will now be explained in greater detail by way of examples and comparative examples, with the understanding that the invention is in no way limited by these examples.

### Examples 1-8 and Comparative Examples 1-8

First, the components shown in Table 1 were mixed to uniformity to obtain primary agents for two-agent hair bleaching compositions (Examples 1-5 and Comparative Examples 1-5). The components shown in Table 2 were also mixed to uniformity to obtain primary agents for two-agent hair dyeing compositions (Examples 6-8 and Comparative Examples 6-8). In Tables 1 and 2, the values in parentheses for polyoxyethylene (15) cetyl ether, polyoxyethylene (2) oleyl ether, polyoxyethylene (50) oleyl ether and polyoxyethylene (30) lanolin represent the addition mole number for ethylene oxide.

Separately, 17 g of a 35 wt % hydrogen peroxide solution, 2 g of cetanol, 0.5 g of sodium lauryl sulfate, 0.02 g of phosphoric acid, 0.1 g of disodium edetate, 0.05 g of phenacetin and 80.33 g of purified water were mixed to uniformity to obtain a secondary agent for the two-agent hair bleaching compositions and two-agent hair bleaching compositions. The pH of the secondary agent was 3.2.

The primary agents for the two-agent hair bleaching compositions of Examples 1-5 and Comparative Examples 1-5 obtained in this manner were used for the following (1) bleaching test while the primary agents for the two-agent hair dyeing compositions of Examples 6-8 and Comparative Examples 6-8 were used for the following (2) dyeing test, and the performance was evaluated.

### (1) Bleaching Test

Just prior to the test, the primary agents for Examples 1-5 and Comparative Examples 1-5 were combined with the secondary agent in a weight ratio of 1:1 to obtain bleaching compositions. Bleaching compositions using the primary agents of Examples 1-5 and bleaching compositions using the primary agents of Comparative Examples 1-5 were coated onto the left and right halves of the top head hair of ten panelists in each of five groups. Also, one 2 g hair bundle of commercially available black hair sample (Chinese hair) was placed on the left and right sides of the head hair and coated with each bleaching composition at the same time as the hair of the panelists. After coating, a vinyl cap was used as a covering to allow bleaching for 30 minutes, and then shampoo was used for washing and a hair dryer was used for drying.

### Evaluation of Scalp Irritation

The degree of scalp irritation felt on both halves of the head during the 30 minutes after coating of the bleaching composition was evaluated on the following 4-level scale.

3: Strong irritation

2: Slight irritation

1: Almost no irritation

0: Absolutely no irritation

The sums of the scores for each group of panelists was divided by the number of panelists (10) and recorded as the evaluation of scalp irritation. The results are shown in Table 1.

### Evaluation of Bleaching Property

The degree of bleaching of the panelists' head hair, i.e. the difference in color between both halves, was judged visually. The results are shown in Table 1.

The lightness (L value) for the black hair sample was measured using an SM Color Computer, Type SM-4 (Suga Test Instruments, Ltd.), and the results are shown in Table 1. Here, a larger L value indicates a lighter hair color, and therefore a greater bleaching effect. The L value for the untreated black hair sample was 16.4.

### (2) Dyeing Test

Just prior to the test, the primary agents for Examples 6-8 and Comparative Examples 6-8 were combined with the secondary agent in a weight ratio of 1:1 to obtain dyeing compositions. Dyeing compositions using the primary agents of Examples 6-8 and dyeing compositions using the primary agents of Comparative Examples 6-8 were coated onto the left and right halves of the top head hair of ten gray-haired panelists in each of three groups. After coating, a vinyl cap was used as a covering before standing for 30 minutes, and then shampoo was used for washing and a hair dryer was used for drying.

### Evaluation of Scalp Irritation

The degree of scalp irritation felt on both halves of the head during the 30 minutes after coating of the dyeing composition was evaluated in the same manner as the scalp irritation evaluation for the bleaching test. The results are shown in Table 2.

### Evaluation of Bleaching and Dyeing Property

The degree of lightness of the panelists' black hair and the degree of dyeing of the panelists' gray hair, i.e. the difference in color tone between both halves, was judged visually. The results are shown in Table 2.

TABLE 1

| | Test group A | | Test group B | | Test group C | | Test group D | | Test group E | |
|---|---|---|---|---|---|---|---|---|---|---|
| | (10 persons) | | (10 persons) | | (10 persons) | | (10 persons) | | (10 persons) | |
| Primary agent composition | | Comp. | | Comp. | | Comp. | | Comp. | | Comp. |
| (g) | Example 1 | Ex. 1 | Example 2 | Ex. 2 | Example 3 | Ex. 3 | Example 4 | Ex. 4 | Example 5 | Ex. 5 |
| Cetyl alcohol | 12 | 12 | 12 | 12 | 12 | 12 | 12 | 12 | 12 | 12 |
| Isopropyl myristate | 5 | 5 | 5 | 5 | 5 | 5 | 5 | 5 | 5 | 5 |
| Polyoxyethylene (15) | 2 | 2 | 2 | 2 | 2 | 2 | 2 | 2 | 2 | 2 |
| cetyl ether | | | | | | | | | | |
| Polyoxyethylene (2) | 2 | 2 | 2 | 2 | 2 | 2 | 2 | 2 | 2 | 2 |
| oleyl ether | | | | | | | | | | |
| Polyoxyethylene (50) | 2 | 2 | 2 | 2 | 2 | 2 | 2 | 2 | 2 | 2 |
| oleyl ether | | | | | | | | | | |
| Polyoxyethylene (30) | 1 | 1 | 1 | 1 | 1 | 1 | 1 | 1 | 1 | 1 |
| lanolin | | | | | | | | | | |
| Coconut oil fatty acid | 1 | 1 | 1 | 1 | 1 | 1 | 1 | 1 | 1 | 1 |
| hydrolyzed collagen/sodium | | | | | | | | | | |
| solution | | | | | | | | | | |
| Ammonia water (28 wt % | 3 | 8 | 4 | 11 | 4 | 4 | 4 | 6 | 4 | 5 |
| concentration) | | | | | | | | | | |
| Monoethanolamine | — | — | — | — | — | — | 1 | — | — | — |
| Sodium hydrogencarbonate | 5 | — | 7 | — | 3 | 4 | 4 | 4 | 4 | 4 |
| Malic acid | 1.5 | — | 1.5 | — | — | — | — | — | — | — |
| Oleic acid | — | — | — | — | — | — | — | — | — | 2 |
| Trisodium citrate | — | — | — | — | 3.3 | — | 3.3 | — | 3.3 | — |
| Sodium hydroxide | 0.5 | — | 0.5 | — | — | 0.5 | — | — | — | 0.5 |
| Anhydrous sodium sulfite | 0.2 | 0.2 | 0.2 | 0.2 | 0.2 | 0.2 | 0.2 | 0.2 | 0.2 | 0.2 |
| Purified water | 64.8 | 66.8 | 61.8 | 63.8 | 64.5 | 66.3 | 62.5 | 64.8 | 63.5 | 63.3 |
| Total | 100 | 100 | 100 | 100 | 100 | 100 | 100 | 100 | 100 | 100 |
| pH of primary agent and | 9.2 | 11.7 | 9.2 | 11.9 | 9.8 | 9.7 | 9.6 | 9.8 | 9.7 | 9.8 |
| dyeing/bleaching composition | | | | | | | | | | |
| Scalp irritation evaluation | 1.0 | 2.1 | 1.4 | 2.7 | 0.9 | 1.8 | 1.1 | 2.0 | 1.0 | 1.9 |
| Difference in degree of | left/right | | left/right | | left/right | | left/right | | left/right | |
| bleaching of panelist head | no difference | | no difference | | no difference | | no difference | | no difference | |
| hair | | | | | | | | | | |
| Results of lightness | 19.5 | 19.4 | 20.5 | 20.5 | 19.4 | 19.3 | 19.7 | 19.7 | 19.4 | 19.4 |
| measurement of black hair | | | | | | | | | | |
| sample (L value: L value for | | | | | | | | | | |
| untreated hair = 16.4) | | | | | | | | | | |

TABLE 2

| | Test group F | | Test group G | | Test group H | |
|---|---|---|---|---|---|---|
| | (10 persons) | | (10 persons) | | (10 persons) | |
| Primary agent | | Comp. | | Comp. | | Comp. |
| composition (g) | Example 6 | Ex. 6 | Example 7 | Ex. 7 | Example 8 | Ex. 8 |
| Paraphenylenediamine | 0.4 | 0.4 | 0.4 | 0.4 | 0.4 | 0.4 |
| Resorcin | 0.3 | 0.3 | 0.3 | 0.3 | 0.3 | 0.3 |
| Paraamino o-cresol | 0.01 | 0.01 | 0.01 | 0.01 | 0.01 | 0.01 |
| Metaaminophenol | 0.05 | 0.05 | 0.05 | 0.05 | 0.05 | 0.05 |
| Cetyl alcohol | 12 | 12 | 12 | 12 | 12 | 12 |
| Isopropyl myristate | 5 | 5 | 5 | 5 | 5 | 5 |
| Polyoxyethylene (15) | 2 | 2 | 2 | 2 | 2 | 2 |
| cetyl ether | | | | | | |
| Polyoxyethylene (2) | 2 | 2 | 2 | 2 | 2 | 2 |
| oleyl ether | | | | | | |
| Polyoxyethylene (50) | 2 | 2 | 2 | 2 | 2 | 2 |
| oleyl ether | | | | | | |
| Polyoxyethylene (30) | 1 | 1 | 1 | 1 | 1 | 1 |
| lanolin | | | | | | |
| Coconut oil fatty | 1 | 1 | 1 | 1 | 1 | 1 |
| acid hydrolyzed | | | | | | |
| collagen/sodium | | | | | | |
| solution | | | | | | |
| Ammonia water (28 | 3 | 8 | 4 | 11 | 4 | 6 |
| wt % concentration) | | | | | | |
| Monoethanolamine | — | — | — | — | 1 | — |
| Sodium | 5 | — | 7 | — | 4 | 4 |
| hydrogencarbonate | | | | | | |
| Malic acid | 1.5 | — | 1.5 | — | — | — |
| Trisodium citrate | — | — | — | — | 3.3 | — |
| Sodium hydroxide | 0.5 | — | 0.5 | — | — | — |
| Anhydrous sodium | 0.2 | 0.2 | 0.2 | 0.2 | 0.2 | 0.2 |
| sulfite | | | | | | |
| Purified water | 64.04 | 66.04 | 61.04 | 63.04 | 61.74 | 64.04 |
| Total | 100 | 100 | 100 | 100 | 100 | 100 |
| pH of primary agent | 9.2 | 11.7 | 9.2 | 11.9 | 9.6 | 9.8 |
| and dyeing/bleaching | | | | | | |
| composition | | | | | | |
| Scalp irritation | 0.9 | 2.4 | 1.2 | 2.6 | 0.9 | 2.3 |
| evaluation | | | | | | |

| | | | |
|---|---|---|---|
| Lightness of black | left/right | left/right | left/right |
| hair | no difference | no difference | no difference |
| Dyeing property on | left/right | left/right | left/right |
| gray hair | no difference | no difference | no difference |

As seen by the results in Table 1, when the primary agents for two-agent hair bleaching compositions of the invention according to Examples 1-5 were used, a high bleaching effect was maintained comparable to that achieved using the primary agents according to Comparative Examples 1-5, which were outside of the scope of the invention, while the scalp irritation was also adequately alleviated.

As seen by the results in Table 2, when the primary agents for two-agent hair dyeing compositions of the invention according to Examples 6-8 were used, a high level of lightness of black hair and dyeability of gray hair were maintained comparable to the level achieved using the primary agents according to Comparative Examples 6-8, which were outside of the scope of the invention, while the scalp irritation was also adequately alleviated.

Consequently, a two-agent hair dyeing/bleaching composition of the invention using a primary agent for a two-agent hair dyeing/bleaching composition according to the invention clearly provides excellent bleaching and dyeing effects while adequately alleviating scalp irritation.

As explained above, by using a primary agent for a two-agent hair dyeing/bleaching composition according to the invention it is possible to obtain a two-agent hair dyeing/bleaching composition with excellent hair bleaching and dyeing effects and adequately reduced scalp irritation, as well as a two-agent hair dyeing/bleaching composition kit suitable for obtaining it.

The hair treatment method of the invention which employs a hair dyeing/bleaching composition according to the invention therefore allows satisfactory bleaching and dyeing of hair with adequately reduced scalp irritation.

From the invention thus described, it will be obvious that the embodiments of the invention may be varied in many ways. Such variations are not to be regarded as a departure from the spirit and scope of the invention, and all such modifications as would be obvious to one skilled in the art are intended for inclusion within the scope of the following claims.

What is claimed is:

1. A primary agent for a two-agent hair dyeing/bleaching composition which comprises a primary agent containing an alkali agent and a secondary agent containing an oxidizing agent, wherein the primary agent contains:
(a) at least one alkali agent selected from the group consisting of ammonia water and monoethanolamine,
(b) at least one water-soluble ammonium salt selected from the group consisting of ammonium carbonate and ammonium hydrogencarbonate, and
(c) at least one first pH adjustor selected from the group consisting of polycarboxylic acids and their salts.

2. A primary agent according to claim 1, wherein the polycarboxylic acids and their salts of said component (c) are citric acid, tartaric acid, malic acid and their salts.

3. A primary agent according to claim 1, which contains the alkali agent of said component (a) at 1-25 wt % (where the ammonia water weight is expressed in terms of ammonia water at a concentration of 28 wt %), the water-soluble ammonium salt of said component (b) at 0.5-20 wt % and the first pH adjustor of said component (c) at 0.1-10 wt %.

4. A primary agent according to claim 1, which further contains (d) at least one second pH adjustor selected from the group consisting of sodium hydroxide, potassium hydroxide and sodium metasilicate.

5. A primary agent according to claim 4, wherein the content of the second pH adjustor of said component (d) is 0.1-8 wt %.

6. A primary agent according to claim 1, wherein the pH is 9.0-12.0.

7. A two-agent hair dyeing/bleaching composition kit provided with
(i) a primary agent for a two-agent hair dyeing/bleaching composition according to claim 1, and
(ii) a secondary agent containing an oxidizing agent.

8. A kit according to claim 7, wherein the polycarboxylic acids and their salts of said component (c) are citric acid, tartaric acid, malic acid and their salts.

9. A kit according to claim 7, which contains the alkali agent of said component (a) at 1-25 wt % (where the ammonia water weight is expressed in terms of ammonia water at a concentration of 28 wt %), the water-soluble ammonium salt of said component (b) at 0.5-20 wt % and the first pH adjustor of said component (c) at 0.1-10 wt %.

10. A kit according to claim 7, which further contains (d) at least one second pH adjustor selected from the group consisting of sodium hydroxide, potassium hydroxide and sodium metasilicate.

11. A kit according to claim 10, wherein the content of the second pH adjustor of said component (d) is 0.1-8 wt %.

12. A kit according to claim 7, wherein the pH is 9.0-12.0.

13. A hair treatment method whereby a hair dyeing/bleaching composition comprising
(a) at least one alkali agent selected from the group consisting of ammonia water and monoethanolamine,
(b) at least one water-soluble ammonium salt selected from the group consisting of ammonium carbonate and ammonium hydrogencarbonate,
(c) at least one first pH adjustor selected from the group consisting of polycarboxylic acids and their salts, and
(e) an oxidizing agent, is applied to hair.

14. A hair treatment method according to claim 13, wherein said hair dyeing/bleaching composition further contains (d) at least one second pH adjustor selected from the group consisting of sodium hydroxide, potassium hydroxide and sodium metasilicate.

15. A hair treatment method according to claim 13, wherein the pH of said hair dyeing/bleaching composition is 9.0-12.0.
